(19) United States
(12) Patent Application Publication
(10) Pub. No.: US 2007/0162019 A1
(43) Pub. Date: Jul. 12, 2007

(54) Resorbable anterior cervical plating system with screw retention mechanism

(76) Inventors: Paul Burns, Perkasie, PA (US); David Koch, Diegten (CH); Christoph A. Roth, West Chester, PA (US)

Correspondence Address: JONES DAY, 222 EAST 41ST STREET, NEW YORK, NY, 10017-6702, US

(21) Appl. No.: 11/643,923

(22) Filed: Dec. 20, 2006

Related U.S. Application Data
(60) Provisional application No. 60/753,372, filed on Dec. 21, 2005.

(51) Int. Cl. A61F 2/30

(52) U.S. Cl. 606/69

(57) ABSTRACT

A bone stabilization or fixation assembly may include a bone plate having an upper side and an underside and at least one fixation hole having a first diameter and the hole extending from the upper side to the underside. The area of the bone plate adjacent the fixation hole has a first thickness. The bone stabilization assembly further includes at least one fastener having a head, a shaft, and a relief. The head has a larger dimension than the shaft. The shaft may have threads having a pitch, a core diameter and an outer thread diameter. The relief has a length and a second diameter. The first diameter of the fixation hole may be smaller than the outer thread diameter but larger than the second diameter of the relief. Also, the length of the relief may be greater than the first thickness of the bone plate. The first diameter may be greater than, equal to, or less than the core diameter of the shaft.

The present application claims priority to U.S. Provisional Application No. 60/753,372 filed on Dec. 21, 2005, the entire contents of which is expressly incorporated herein by reference.

### TECHNICAL FIELD

The present invention is directed to a bone stabilization or fixation assembly, particularly for use in the spine.

### BACKGROUND OF THE INVENTION

Orthopedic fixation devices are frequently coupled to bone by the use of fasteners, such as screws or pins. For example, bone plates can be secured to bone with screws inserted through plate holes. In the past, many of the orthopedic devices were made primarily from metallic materials. The metallic devices have some advantages, such as the ability to sterilize and having the necessary strength for support and fixation. However, the metallic devices have their disadvantages, such that when the bone defect is repaired the device either remains in the body or is surgically removed.

More recently, improved materials, including non-metallic devices, have been used to treat bone defects. The non-metallic devices can remain in the body, or the device may be made of materials that are biodegradable over time. A disadvantage of these non-metallic devices is that they do not provide sufficient mechanical strength or holding force such that the fasteners may become dislodged or backout. Further, these devices may not be visible during imaging of a patient, such as in X-rays. The current state of the art for orthopedic devices, and in particular anterior cervical plating systems, is to retain the fastener within the plate thus preventing screw backout and subsequent esophagus irritation and/or Dysphagia. Current systems that employ such a mechanism typically are produced from metal (Ti or Ti Alloy).

### SUMMARY OF THE INVENTION

It is an object of the stabilization or bone fixation assembly (fasteners and plates) preferably to be resorbable and employ a fastener retention mechanism. The bone stabilization or fixation assembly comprises a bone plate having an upper side and an underside and at least one fixation hole having a first diameter and the hole extending from the upper side to the underside. The bone plate adjacent the fixation hole has a first thickness. The bone stabilization assembly preferably further comprises at least one fastener having a head, a shaft, and a relief. The head preferably has a larger width dimension than the shaft. The shaft may have threads having a pitch, a core diameter and an outer thread diameter. The relief has a length and a second diameter. The second diameter of the relief is preferably equal to or less than the core diameter of the shaft. The length of the relief may be greater than the first thickness of the bone plate. The first diameter of the fixation hole also may be smaller than the outer thread diameter but larger than the second diameter of the relief. The first diameter of the fixation hole may be greater than, equal to, or less than the core diameter of the shaft.

In one embodiment, the bone plate and fasteners preferably are composed of resorbable polymers and plastics, such as for example, 70/30 (L/DL) Polylactide. Other materials such as for example, magnesium alloys, titanium, and stainless steel are also contemplated.

Radiopaque marker beads preferably are inserted into recesses near or at the edges of the bone plate. The bone plate may include two, three or more pairs of fixation holes. Instead of grouping the fixation holes by pairs, single fixation holes may also be used. Other configurations of bone plate fixation holes, however, are contemplated. The diameter of the fixation holes at the upper side of the bone plate may be larger than at the lower or under side of the bone plate. The bone plate may include at least one slot for receiving a drill/screw guide, for graft visualization or both. The fasteners may include marker beads near proximal and distal ends of the shaft, and recesses may be formed in the fastener to accommodate insertion of the marker beads.

In another embodiment, a bone fixation assembly comprises a bone fixation device having at least one aperture configured to receive a bone fastener. The aperture may have a diameter that is smallest at an underside of the bone fixation device. The region of the bone plate adjacent the fixation hole has a first thickness. The bone fixation assembly also includes a bone fastener with threads receivable in the aperture in an installed position. The threads have a pitch and an outer thread diameter. The fastener may also include a relief portion with a first length and a first diameter. The first diameter of the relief portion preferably may not be greater than the smallest diameter of the aperture. The outer thread diameter may be greater than the smallest diameter of the aperture, and the length of the relief portion preferably may be longer than the first thickness.

A method of fixing a bone plate to a bone includes selecting the bone plate having an upper side, an underside, and at least four plate fixation holes extending from the upper side to the underside. The fixation holes have a diameter that is smallest at the underside of the bone plate. The region of the bone plate adjacent the fixation holes has a first thickness. The method further includes drilling and tapping the bone plate for inserting at least two bone fasteners having a head and a shaft with threads into at least two of the plate fixation holes. The fasteners further include a relief portion having a first length and a first diameter. The method further includes verifying screw retention visually or by tactile feedback. The outer thread diameter may be greater than the smallest diameter of the fixation hole and the first diameter of the relief portion may not be greater than the smallest diameter of the fixation holes. The length of the relief portion may be greater than the first thickness such that when the at least two bone fasteners are fully seated in the bone plate, the fasteners disengage from the bone plate.

In a further embodiment, a bone fixation assembly comprises a bone fixation device having an aperture configured to receive a bone fastener and an aperture boundary surrounding the aperture. The assembly further includes a bone fastener receivable in the aperture in an installed position. The fastener may have a shaft portion, a head portion, and a retainer portion extending radically outward and configured to engage the bone fixation device at the boundary of the aperture to restrain withdrawal of the bone screw from the installed position.

In a further embodiment, the retainer portion of the fastener includes a flange that is resiliently deflectable radially inward upon moving axially through the aperture in the bone fixation device.

In a further embodiment, the retainer portion of the fastener includes circumferenctially spaced sectors of the fastener that are resiliently deflectable radially inward upon moving into the aperture in the bone fixation device.

In another embodiment, the bone fastener includes a body portion defining a head and a threaded stem projecting axially from the head, and the retainer portion comprises a split ring mounted on the head.

In still another embodiment, the bone fastener is configured to be received in the aperture in an installed position. The head is configured to receive a driving tool, and the retainer structure is configured to deflect into installed engagement with the bone fixation device to block removal of the fastener from the aperture. The retainer structure is connected to at least one of the shaft and the head and movable toward and into the installed position as a unit that is separate from the bone fixation device.

The bone fixation assembly may have applications in the spine in the cervical and lumbar regions, including for example anterior cervical plating, and employ a retention mechanism. This retention mechanism has at least the advantage of providing for the following:

(1) The screws may translate and toggle relative to the plate allowing the vertebral bodies to settle, thus maintaining a compressive load on the graft and promoting fusion.

(2) The screws can be inserted at variable angles providing the surgeon options in screw placement. The screw angle may be controlled by a drill guide which may keep the angle within a specified tolerance zone, e.g., about ±20°.

(3) Preferably, screw retention can be verified post-insertion visually or by tactile feedback.

(4) Preferably, the increase in screw insertion torque due to the retention mechanism is independent of the torque increase due to lagging the screw to the plate, i.e., the surgeon will not confuse engagement of the screw retention with tightening the screw against the plate.

Further objects, features, aspects, forms, advantages, and benefits shall become apparent from the description and drawings contained herein.

### BRIEF DESCRIPTION OF THE DRAWINGS

The bone fixation assembly is explained in even greater detail in the following exemplary drawings. The drawings are merely exemplary to illustrate the structure, operation and method of use of the bone fixation assembly and certain features that may be used singularly or in combination with other features and the invention should not be limited to the embodiments shown.

FIG. 1 shows a perspective view of an embodiment of the bone fixation assembly,

FIG. 2 shows a perspective view of the bone plate of the embodiment depicted in FIG. 1,

FIG. 3 is a top view of the bone plate of the embodiment depicted in FIG. 1,

FIG. 4 is a bottom view of the bone plate of the embodiment depicted in FIG. 1,

FIG. 5 is a side view of the bone plate of the embodiment depicted in FIG. 1,

FIG. 6 is a cross-sectional side view of the bone plate of the embodiment depicted in FIG. 1,

FIG. 7 is a perspective view of an alternative embodiment of the bone plate of the embodiment depicted in FIG. 1,

FIG. 8 is a perspective view of the fastener depicted in FIG. 1,

FIG. 9 is a cross-sectional view of the fastener of FIG. 8,

FIG. 10 is a cross-sectional view of the assembly depicted in FIG. 1,

FIG. 11 is a cross-sectional view of another embodiment of a bone fixation assembly,

FIG. 12 is a perspective view of the assembly depicted in FIG. 11,

FIG. 13 is a perspective view of a third embodiment of the bone fixation assembly,

FIG. 14 is a cross-sectional perspective view of the embodiment depicted in FIG. 13,

FIG. 15 is a side cut-out view of a fourth embodiment of the bone fixation assembly, and

FIG. 16 is a perspective view of the assembly depicted in FIG. 15.

### DESCRIPTION OF THE PREFERRED EMBODIMENTS

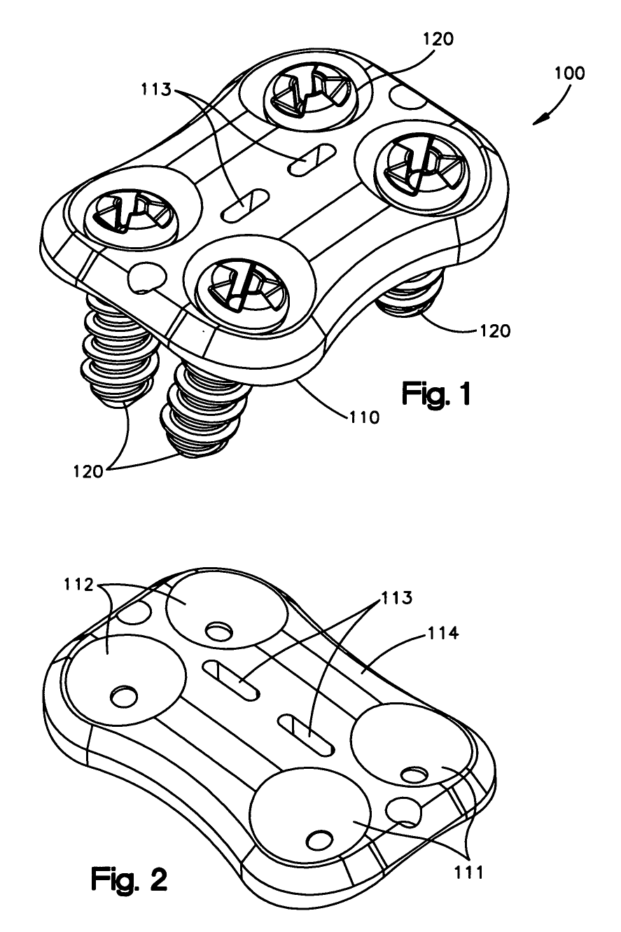

A preferred embodiment of a bone plate assembly 100 (also referred to as a bone fixation assembly or bone stabilization assembly) is depicted in FIG. 1, and includes a bone plate 110 and fasteners 120. The bone plate assembly is preferably for use in the human spine, preferably in the cervical and/or lumbar regions. The bone plate assembly may be attached, for example, to two or more adjoining vertebrae and functions to prevent graft extrusion/expulsion. With proper strength of the bone plate, the bone plate assembly may also provide stability for alignment and maintaining adjacent vertebrae in a predetermined spatial relationship to each other. The bone plate assembly may be used for other regions other than the spine, such as for example, long bones.

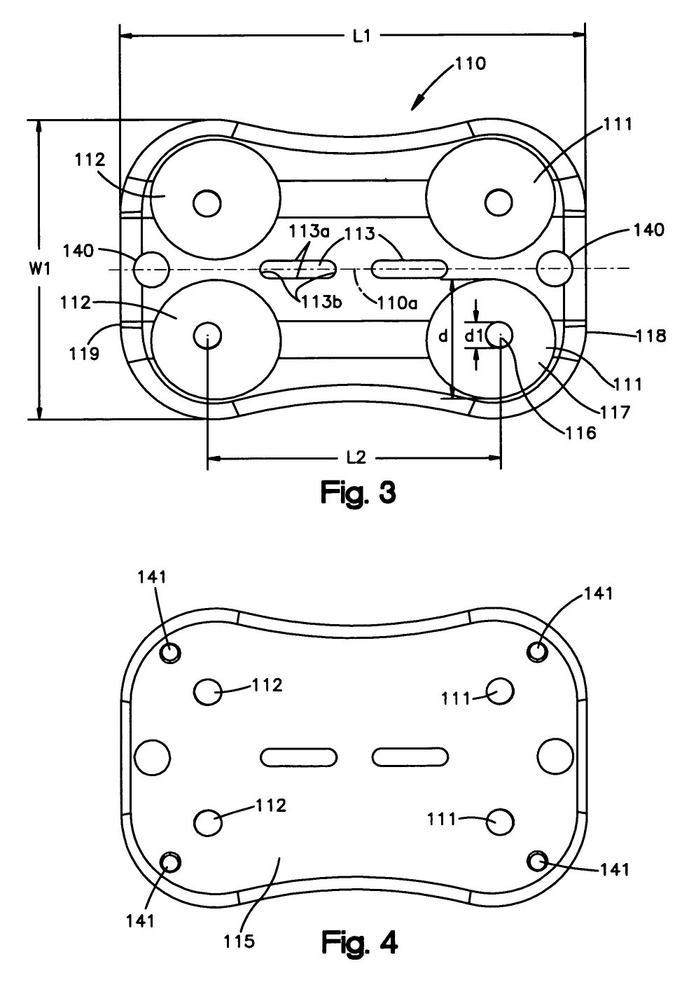

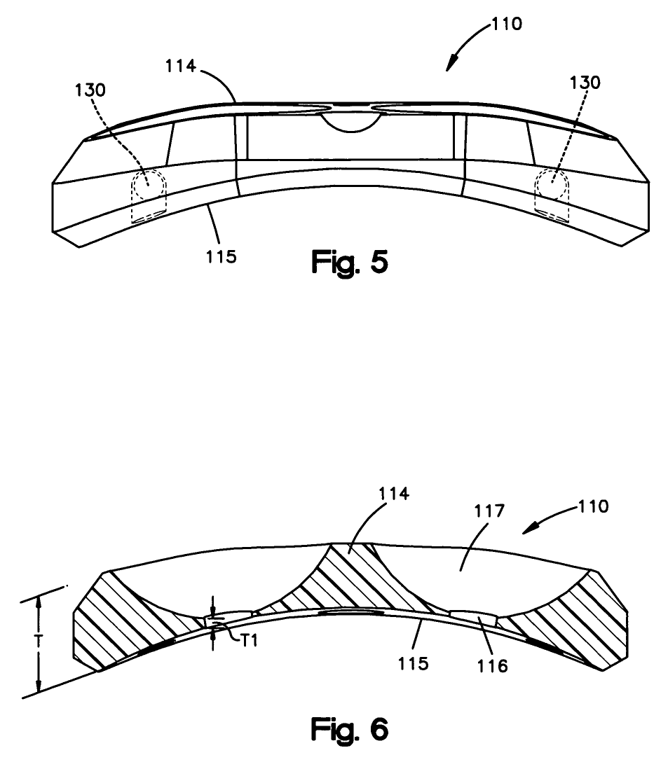

Bone plate 10 (FIGS. 2-6) includes an upper side 114 (FIG. 3) and an underside or lower side 115 (FIG. 4) with fixation holes extending from the upper side 114 to the underside 115. The underside 115 may be curved transverse to the central longitudinal axis 110a. The upper side 114 may also be curved (FIG. 5). Bone plate 110 represents a one level implant for attaching to two adjacent vertebrae and may have an overall length L1 of between about 18.0 mm and about 36.0 mm, a width W1 of between about 6 mm and about 20 mm, preferably about 15 mm, and a midline thickness between about 1 mm and about 6 mm, preferably about 2.0 mm. Various size plates could be offered where the lengths can increase in 2 mm increments. Other incremental sizes are contemplated. Likewise the width and thickness may changes as the length increases. Bone plate 110′ (FIG. 7) represents a two level implant for attaching to three adjacent vertebrae and may have an overall length L3 of between about 36.00 mm and about 56.00 mm, a width W2 of between about 6 mm and about 20 mm, preferably about 15 mm, and a thickness T of between about 1 mm and about 6 mm, preferably about 2.0 mm. The dimensions of the bone plate 110, 110′ are not limited by the values noted, and may be dependent upon the anatomical characteristics of the patient. Other sizes and levels of implants are also contemplated. The bone plate 110 preferably may be composed of a resorbable material or resorbable plastic, such as for example 70/30 (L/DL) Polylactide. Other polymers and plastics, as well as resorbable metals such as magnesium alloys, and metals such as titanium, stainless steel, etc. are also contemplated for the bone plate. It is also contemplated that the bone plate 110, 110′ may be made of a metal with resorbable, molded inserts about the diameter of the fixation holes. This may permit drilling and tapping of the resorbable inserts, in situ or during the surgical procedure.

The bone plate 110 may include two or more pairs of fixation holes, first pair of fixation holes 111, and second pair of fixation holes 112. The fixation holes 111, 112 may be circular in shape and extend from the upper side 114 to the underside 115. The fixation hole opening 117 on the upper side 114 is concave in shape (FIG. 6) and has a diameter d (FIG. 3) which is greater than a minimum diameter d1 of the fixation hole opening (minimum diameter hole) 116 on the underside 115 of the plate 110. The minimum diameter d1 preferably is between about 1.0 mm and about 6.0 mm, and more preferably about 2.9 mm. The minimum diameter hole 116 may or may not be centered in the concave portion 117. The distance L2 between the minimum diameter holes 116 of the fixation holes 111, 112 along the central longitudinal axis 110a for a one level implant is between about 11.5 mm and about 25.5 mm depending on the overall length L1 of the implant. For a two level implant, such as depicted in FIG. 7, the distance L4 between the outer most minimum diameter holes 116 of the fixation holes 111, 112 along the central longitudinal axis 110a is between about 25.70 mm and about 45.70 mm depending on the overall length L3 of the implant. The thickness TI of the bone plate 110 near or adjacent the minimum diameter hole 116 is preferably about 1 mm depending on the thickness T of the bone plate 110. However, because the bone plate 110 may drilled and/or tapped to create the final fixation holes 111, 112, the thickness T1 may be any dimension and preferably permits the fastener 120 to disengage with the bone plate 110 when the fastener is fully inserted through the bone plate 110. It is also contemplated that the bone plate may be only tapped with a self-drilling tap. Further details regarding the relationship between the bone plate, fixation holes, and the fasteners will be described later. Although the fixation plate 110 is provided with two pairs of fixation holes 111, 112, more than two pairs of fixations holes may be provided (FIG. 7), for example, so that the plate 110 may span a greater length and thus be fastened to multiple locations along the spine. Alternatively, single holes may be provided as opposed to pairs of fixation holes.

At least one slot 113 may be aligned along central longitudinal axis 110a for receiving a drill/screw guide, for graft visualization or for both. Preferably, slot 113 does not receive any fasteners. In alternative embodiments, more than one slot may be provided (as shown), and the slot or slots may be disposed transverse to the central longitudinal axis 110a. Preferably, slot 113 includes straight portions 113a and semicircular portions 113b, although other shapes for slot 113 are contemplated. Additional plate holes 140 may be located at the ends 118, 119 of the plate along the central longitudinal axis 10a for visualization and/or receiving instruments.

The under side 115 of the bone plate 110 may include recesses 141 near or at the corners of the bone plate 110. The recesses are dimensioned to allow for marker beads 130 to be inserted (FIG. 5), and may have a depth preferably of 1.1 mm depending on the thickness of the bone plate 110. The marker beads 130 preferably do not extend beyond the opening of the recesses 141. These marker beads 130 are radiopaque and allow identifying the corners of the plate during imaging. The marker beads 130 may be composed of tantalum, however other materials are contemplated. The recesses and markers may be provided at alternative or additional locations.

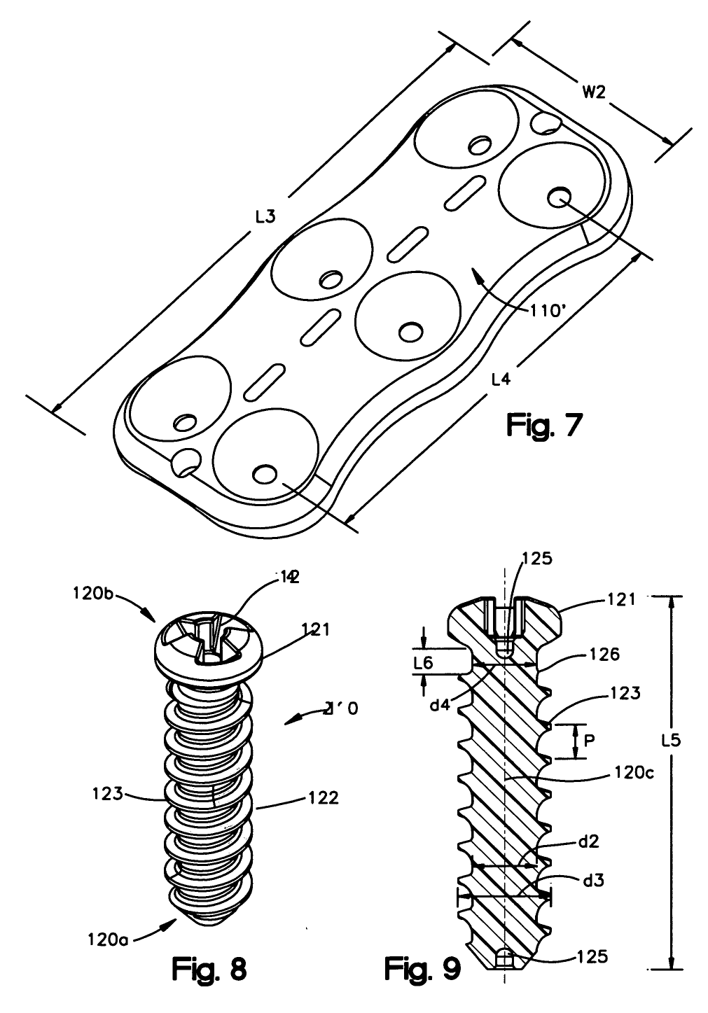

The fasteners 120 (FIGS. 8 and 9) have a distal end 120a, a proximal end 120b, and a longitudinal axis 120c. The fasteners preferably have a head 121 at the proximal end 120b, a shaft 122 with threads 123, and a relief region 126 adjacent the head 121. The relief region 126 may be substantially smooth and devoid of threads and preferably permits the fastener 120 to disengage from the plate when the fasteners 120 are fully seated through the bone plate, thereby minimizing the load on the fastener/plate interface and subsequently the plate. This has the advantage of minimizing post operative failure of the implant due to the load on the plate. Furthermore, disengagement of the fasteners from the plate 110 may allow the fasteners to toggle post operatively, thereby allowing the vertebral bodies to settle and maintain a compressive load on the graft for accelerated bone growth and better fusion.

The fastener 120 may have an overall length L5 of between about 8 mm and about 40 mm. The head 121 preferably has a larger diameter than the core diameter d2 of the shaft. The core diameter of the shaft d2 may be between about 1.0 mm and about 5.0 mm, preferably about 2.8 mm. The threads 123 have a pitch (i.e., the distance between respective threads) P of between about 0.5 mm and about 2.5 mm, preferably about 1.5 mm, and an outer thread diameter d3 of between about 2 mm and about 6 mm, and preferably about 4.0 mm. The relief diameter d4 is independent of the core diameter d2 and thus may be greater than, but preferably is equal to, or smaller than the core diameter a core diameter d2. The relief diameter d4 may be between about 1.0 mm and about 5.0 mm, and preferably is about 2.8 mm. Further, the relief region 126 has a length L6 of between about 0.2 mm and about 3.0 mm, and preferably about 0.8 mm. The dimensions of the fasteners 120 are not limited by the values noted. Other sizes are also contemplated. The fasteners 120 may be composed of a resorbable material or resorbable plastic, such as for example 70/30 (L/DL) Polylactide. Other polymers and plastics, as well as resorbable metals such as for example magnesium alloys and metals such as for example titanium, stainless steel, etc. are also contemplated for the fasteners.

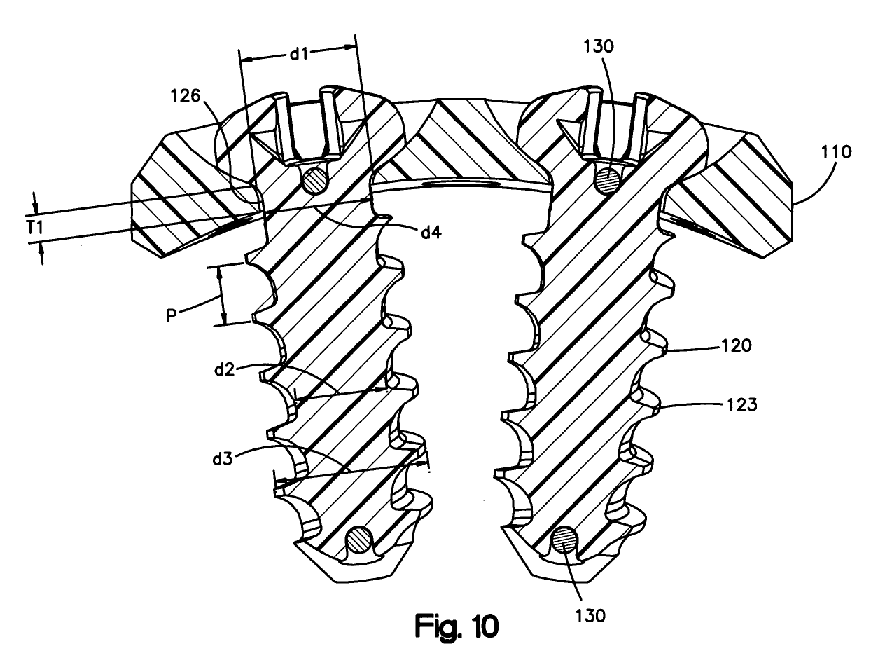

The head 121 of fastener 120 is configured to have a tool-engaging structure 124 for receiving a driving tool (not shown). The tool-engaging structure 124 may be compatible for receipt of a Phillips-type driving tool. The specific tool-engaging structure is not critical; accordingly it is within the scope of the embodiment to include fasteners having various tool-engaging structures associated with the head 121. In addition, the head 121 may include a recess 125 along the longitudinal axis 120c and into the shaft 122 that is dimensioned to allow for a radiopaque marker bead 130 (FIG. 10) to be inserted. The distal end 120a of the fastener 120 may also include a recess 125 dimensioned to allow for a radiopaque marker bead 130 (FIG. 10) to be inserted. The size of the recess 125 at both the distal and proximal ends may vary, but is preferably about 0.9 mm.

In one embodiment, the fastener 120 may be prevented from backing out axially by interference between the bone plate 110 and the faster threads 123 due to a relationship between the bone plate 110 and the fasteners 120, as shown in FIG. 10. If a fastener 120 tries to back out through linear translation, the threads 123, having an outer thread diameter d3, are blocked by the bone plate, having a minimum diameter d1 in the area of the fixation holes, because the outer thread diameter d3 is larger than the minimum diameter d1 of the fixation holes 111, 112. The fastener 120, however, can be inserted or threaded through the fixation holes 111, 112 because diameter d1 is approximately equal to or greater than the core diameter d2 of the shaft 122. It is also contemplated that a fastener having a core diameter d2 larger than the minimum diameter d1 of the fixation holes 111, 112 may be used. In such an embodiment, the relief diameter d4 may be smaller than the core diameter d2 and smaller than the minimum diameter d1 of the fixation holes 111, 112 to allow the plate 110 and fastener 120 to disengage from one another. For example, there may exist a 0.3 mm interference between the core diameter d2 and the minimum diameter d1 (d2>d1), such that as the fastener is threaded/pushed through the fixation hole, the fixation hole, being resorbable expands slightly to accommodate the larger core diameter d2 of the fastener. In a representative example of such an embodiment, the core diameter d2 of the fastener may be 2.8 mm and the minimum diameter d1 of the fixation hole may be 2.5 mm, and the relief diameter may be equal to or less than 2.5 mm.

The fasteners 120 preferably may be inserted at various angles to the plate 110. The surgeons may use a drill guide to determine the desired fastener angle with respect to the bone plate. Preferably, at the desired fastener angle the bone plate maintains a full 360 degree retention around the fastener. The angle of the fastener with respect to the bone plate may be up to 20 degrees off from vertical with respect to the bone plate, although angles greater than 20 degrees are contemplated. The concavity of the fixation hole opening 117 on the upper side 114 of the bone plate 110 in which the head 121 of the fastener 120 is seated when the fastener is fully threaded through the plate is dimensioned to allow the fastener to be inserted at an angle, and also permit the fastener to change angle with respect to the bone plate over time as the vertebrae compress, a feature referred to as toggling or fastener toggle. After insertion, screw retention may be verified visually or by tactile feedback.

In one embodiment, the thickness of the plate in the region where the hole diameter is less than the thread diameter is preferably less than the pitch of the fastener. This relationship may have benefit where the fasteners and bone plate are metal. In a bone plate, where the fixation hole drilled and/or tapped, the thickness TI of the bone plate near minimum diameter d1 of the fixation hole 111, 112 may be any dimension although it is preferred that the fastener disengage from the plate 110. In this embodiment, there need be no relationship between plate thickness T1 and the thread pitch P, such that thickness Ti may be greater than, equal to, or less than the thread pitch P. This feature may have particular application in polymeric or in plastic plates where the fixation hole may be drilled and/or tapped during the surgical procedure. Where the thread diameter of the fastener is larger than the fixation hole, tapping the fixation hole, preferably the polymer or plastic material surrounding the fixation hole, permits the fastener to pass through the fixation hole preferably without deforming the fastener or the bone plate. The fixation hole is preferably drilled and/or tapped at the desired insertion angle for the fastener. As noted, the plate may be drilled and then tapped, using two separate instruments. However, it is also contemplated that the plate may be only tapped with a self-drilling tap.

The dimensions of the relief region 126 and the dimensions of the fixation holes 111, 112, specifically the minimum diameter d1 and thickness TI near or about the minimum diameter hole 116 controls the amount of toggle between the fastener 120 and the bone plate 110. The plate thickness TI near or about the minimum diameter d1 of the fixation hole preferably is less than the length L6 of relief region 126, and the degree of toggle may be controlled by this relationship, as well as the relationship between the relief diameter d6 and the minimum diameter d1. That is, the longer the relief length L6 is with respect to the thickness T1 the more the fastener may toggle with respect to the bone plate. Similarly, the greater the difference between the diameter d6 of the relief portion and the minimum diameter d1 of the fixation hole, the greater the amount of toggle that can be obtained. Conversely, the larger the relief diameter d6 is to the minimum diameter d1 of the fixation hole 111, 112 and/or the shorter the length L6 of the relief 126 is to thickness TI the less able the fastener 120 will be able to toggle with respect to the bone plate 110.

Other embodiments of a bone fixation assembly will now be described. Although, different reference designators are used to describe the bone plate and fasteners of the various embodiments, only differences in these components will be described, specifically the interface between the bone plate and the fasteners. Other elements, for example marker beads, are the same or similar and will not be described further.

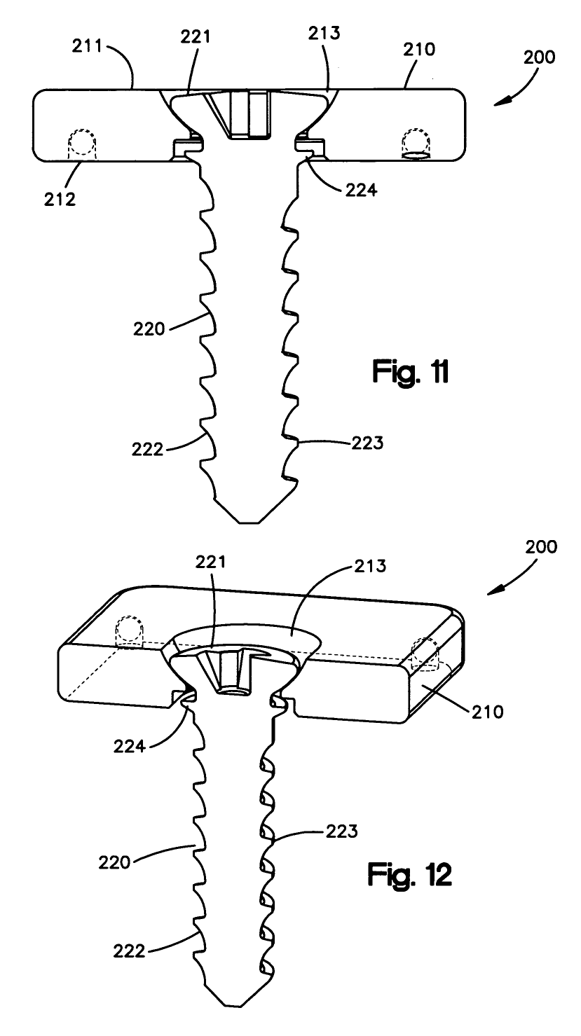

In another embodiment, the bone fixation assembly 200, shown in FIGS. 11 and 12, may comprise a bone plate 210 having an upper side 211, an underside 212 facing the bone, and at least one fixation hole 213 having a diameter D′. The bone fixation assembly 200 also may include fasteners 220 having a head 221, a shaft 222 with threads 223, and a flange 224 having an outer diameter D located near the head 221 of the fastener 220. The outer diameter D of the flange 224 is larger than the minimum diameter or at least a portion of the fixation hole 213 so that during insertion of the fastener 220, the flange 224 deflects to a smaller dimension as it passes through the fixation hole 213 and then expands, preferably to its un-deformed shape and dimension after it passes through the bone plate 210. The flange preferably undergoes elastic deformation as it is inserted through the fixation hole 213 although some plastic deformation may also occur. The flange 224 preferably is stiffer in the reverse direction such that the fastener 210 is restricted from backing out.

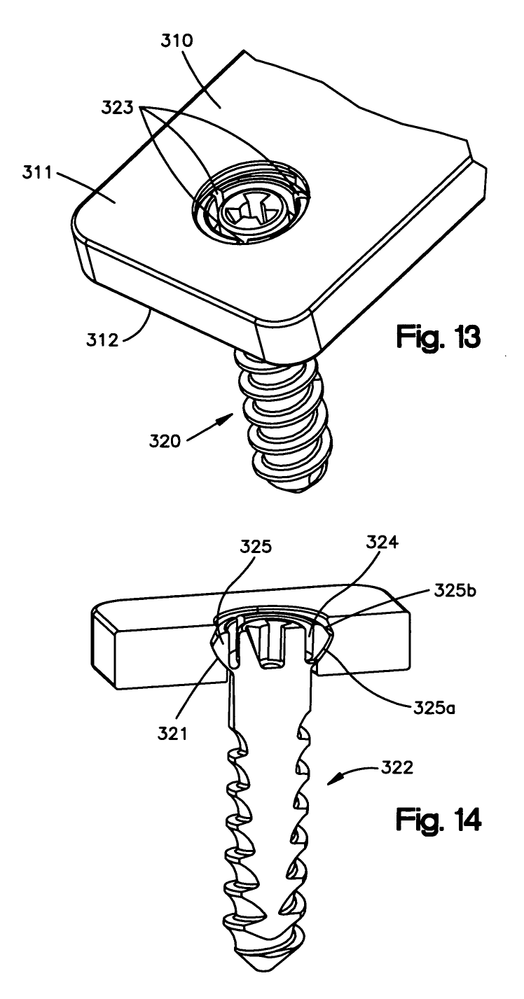

FIGS. 13 and 14 depict another embodiment of a bone fixation assembly 300. This embodiment similarly includes a bone plate 310 and fasteners 320. The bone plate 310 may have an upper side 311, an underside 312 facing the bone, and at least one fixation hole 313. The opening to the plate fixation hole 313 on both the upper side 311 and the underside 312 are of a diameter that is smaller than the diameter of the fixation hole in between the two openings, preferably creating a cross-section for the fixation hole that has a curved contour (See FIG. 14) or two sections that taper as they approach the upper side and underside of the bone plate. The contour of the plate fixation hole 313 may have a cutout that correspondingly mates with the head 321 of the fastener 320. The fastener 320 may have a head 321, and a shaft 322 with threads. The fastener 320 may employ a flexible head 321 that deflects radially inward as the head 321 enters the plate fixation hole 313. An expandable flange 325 associated with the head 321 may have one or more slots 323 (FIG. 13 depicts three slots). The expandable flange 325 in conjunction with an annular groove 324 allow the head 321 to deflect. The expandable flange preferably has a first region 325a that is tapered or contoured so that the diameter of the head 321 increases from the shaft toward the top of the head. The flange preferably has as second region 325b that is tapered, contoured, or provides a shoulder so that the diameter preferably decreases towards the top of the fastener. Thus, as the fastener head 321 is inserted through the hole 313 the edge or portion of the bone plate smaller than the flange deflects the flange inward as the fastener moves through the hole. As the fastener continues through the fixation hole, the flange expands to fill the corresponding cutout in the plate, preventing the fastener 320 from backing out of the plate fixation hole 313.

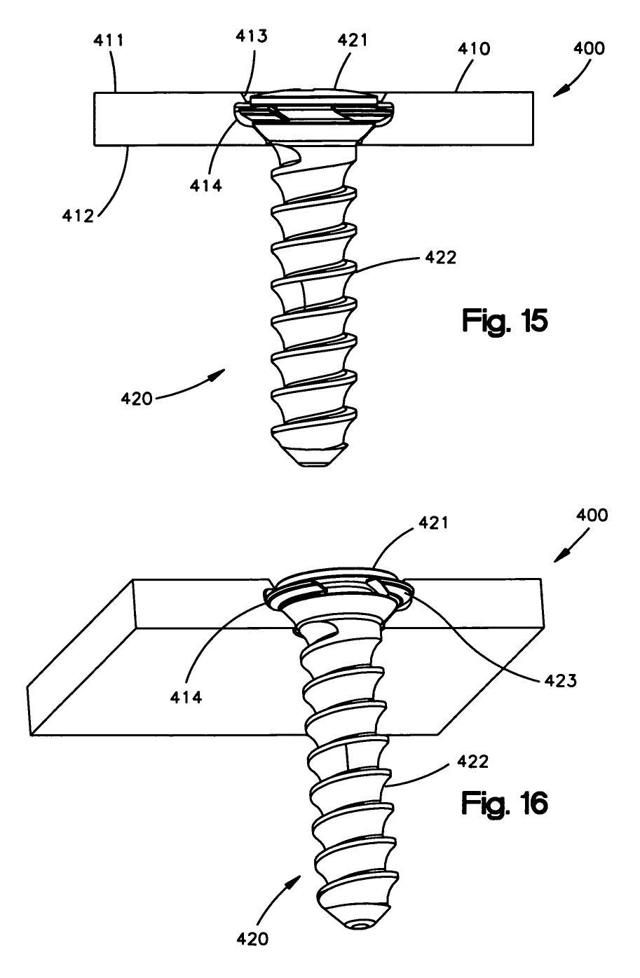

FIGS. 15 and 16 depict another embodiment of a bone fixation assembly 400. This embodiment similarly includes a bone plate 410 and fasteners 420. The bone plate 410 may have an upper side 411, an underside 412 facing the bone, and at least one plate fixation hole 413. The plate fixation hole 413 may include a plate groove 414, such that the diameter of the openings on the upper side 411 and underside 412 is smaller than the diameter of the plate groove 414. The fastener 420 may include a head 421 and a shaft 422 with threads. The head 421 of the fastener may include a flexible annular ring 423 such that the annular ring 423 is compressed and closes as the screw head 421 enters the plate fixation hole 413. The flexible, annular ring 423 expands back to its original size as it enters the plate groove 414, thereby preventing the screw from linearly backing out. The fastener is prevented from backing out because the flexible, annular ring 423 is secured within the plate groove 414.

It is contemplated that the features of the above embodiments of the bone fixation assembly may be combined in a number of combinations to produce derivative embodiments. Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A bone plate assembly comprising:
a bone plate having an upper side and an underside and at least one fixation hole having a first diameter, the fixation hole extending from the upper side to the underside, wherein an area of the bone plate adjacent the fixation hole has a first thickness; and
at least one fastener, the fastener having a head, a shaft, and a relief region, the head having a larger width than the shaft, the shaft having a core diameter and threads, the threads having a pitch, and an outer thread diameter, and the relief region having a length and second diameter,
wherein the first diameter is smaller than the outer thread diameter but larger than the second diameter, and
wherein the length of the relief is greater than the first thickness of the bone plate.

2. The bone plate assembly according to claim 1, wherein the bone plate and fasteners are composed of plastic or polymer material.

3. The bone plate assembly according to claim 2 wherein the polymer material is 70/30 (L/DL) Polylactide.

4. The bone plate assembly according to claim 1 wherein the fasteners are composed of magnesium alloys.

5. The bone plate assembly according to claim 1, wherein the bone plate further comprises radiopaque marker beads.

6. The bone plate assembly according to claim 1, wherein the bone plate includes two pairs of fixation holes.

7. The bone plate assembly according to claim 1, wherein the bone plate includes at least three pairs of fixation holes.

8. The bone plate assembly according to claim 1, wherein the fixation hole on the upper side has a concave shape and a diameter larger than at the underside of the bone plate.

9. The bone plate assembly according to claim 1, wherein the bone plate includes at least one slot for receiving a drill/screw guide or for graft visualization.

10. The bone plate assembly according to claim 1, wherein the fastener includes radiopaque marker beads near proximal and distal ends of the shaft.

11. The bone plate assembly according to claim 1, wherein the bone plate is curved about a central longitudinal axis.

12. A bone plate assembly comprising:
a bone fixation device having an upper side, an underside and at least one aperture configured to receive a bone fastener, the aperture having a smallest diameter d1 and wherein a region of the bone plate adjacent the aperture has a first thickness; and
a bone fastener having a head with a diameter, threads with a pitch and an outer thread diameter, and a relief region with a first length and a first diameter and no threads,
wherein the outer thread diameter is greater than the smallest diameter d1 of the aperture, and the length of the relief region is greater than the first thickness.

13. The bone plate assembly of claim 12, wherein the head diameter of the fastener is greater than the outer thread diameter.

14. The bone plate assembly of claim 12, wherein the first diameter is less than the diameter of the head.

15. A method of fixing a bone plate to a bone, the method comprising:
selecting the bone plate having an upper side, an underside, and at least four plate fixation holes extending from the upper side to the underside, the fixation holes having a diameter that is smallest between the underside and the upper side of the bone plate and wherein an area of the bone plate adjacent the fixation hole has a first thickness;
drilling and tapping the bone plate for inserting at least two bone fasteners;
inserting at least two bone fasteners having threads with a pitch and an outer thread diameter and a relief region with a first length and first diameter through at least two of the plate fixation holes; and
verifying screw retention visually or by tactile feedback,
wherein the outer thread diameter is greater than the smallest diameter of the fixation hole, and
wherein the first diameter is not greater than the smallest diameter of the fixation hole and the length of the relief region is greater than the first thickness such that when the at least two bone fasteners are fully seated in the bone plate the fastener disengages from the bone plate.

16. A bone fixation assembly comprising:
a bone fixation device having an aperture configured to receive a bone fastener and
an aperture boundary surrounding the aperture; and
a bone fastener receivable in the aperture in an installed position, the fastener having a shaft portion, a head portion, and a retainer portion extending radically outward and configured to engage the bone fixation device at the boundary of the aperture to restrain withdrawal of the bone screw from the installed position.

17. The bone fixation assembly according to claim 16 wherein the retainer portion of the bone fastener comprises a flange that is resiliently deflectable radially inward upon moving axially through the aperture in the bone fixation device.

18. The bone fixation assembly according to claim 16, wherein the retainer portion of the bone fastener comprises circumferenctially spaced sectors of the fastener that are resiliently deflectable radially inward upon moving into the aperture in the bone fixation device.

19. The bone fixation assembly according to claim 16, wherein the bone fastener includes a body portion defining a head and a threaded stem projecting axially from the head, and the retainer portion comprises a split ring mounted on the head.

20. The bone fixation assembly according to claim 16, wherein the bone fastener is configured to be received in the aperture in an installed position, the head is configured to receive a driving tool, and the retainer structure is configured to deflect into installed engagement with the bone fixation device to block removal of the fastener from the aperture, with the retainer structure being connected to at least one of the shaft and the head and movable toward and into the installed position as a unit that is separate from the bone fixation device.
